(19) United States
(12) Patent Application Publication
(10) Pub. No.: US 2026/0062741 A1
(43) Pub. Date: Mar. 5, 2026

(54) POLYMER ASSISTED GENOMIC ANALYSIS

(71) Applicant: DHR. VOLKER LEEN, Kortenaken (BE)

(72) Inventor: Volker LEEN, Kortenaken (BE)

(21) Appl. No.: 18/820,719

(22) Filed: Aug. 30, 2024

(51) Int. Cl. C12Q 1/6869; C12Q 1/6883; G01N 21/64

(52) U.S. Cl. CPC C12Q 1/6869; C12Q 1/6883; G01N 21/6458

(57) ABSTRACT

Methods and compounds for the analysis of biomolecules are disclosed. The method includes combining a specific marker of a biomolecule with a polymer capable of increasing in size, thus providing access to enhanced information about the structure and identity of the biomolecule.

### TECHNICAL FIELD OF THE INVENTION

Embodiments herein relate to the grafting of biomolecules onto a polymer matrix, where the polymer and its physical behavior enable genomic analysis

### REFERENCES

Gottfried A. et al. “Sequence-specific covalent labeling of DNA,” Biochemical Society Transactions, 39(2), 623-628
Compounds and processes for single-pot attachment of label to nucleic acid, US2006/0188927
Proudnikov D., et al, (1996), Chemical methods of DNA and RNA fluorescent labeling, Nucleic Acids Research,, Vol. 24, 4535-4532
Biomolecular marking, U.S. Pat. No. 6,657,052 B1
Selection of single nucleic acids based on optical signature, US2014/0011686
Methods for specific labeling of nucleic acids with CRISPR/CAS, US 2016/0168621
Methods and devices for whole genome analysis based on single molecules U.S. Pat. No. 8,628,919
Covalent labeling of nucleic acids, Nils Klöcker, Florian P. Weissenboeck and Andrea Rentmeister; 10.1039/D0CS00600A, Chem. Soc. Rev., 2020, 49, 8749-8773.

### BACKGROUND OF THE INVENTION

The study of biopolymers is greatly facilitated by methods that exploit the spatial organization of specific monomers or specific combinations thereof, with the goal of locating the presence, position, abundance or sequence of such specific sites. Examples of such specific sites may include individual nucleobases, amino acid side chains or combinations thereof.

Examples of methods that enable such observations of sequence-specific domains include Fiber-FISH and Genomic Mapping, both of which have extensive applications in human diagnostics.

In the pursuit of longitudinal information, these approaches quickly reach their limits when it comes to distinguishing a large number of sequence-specific domains, where the resolution of the underlying sequence information is inadequate. Moreover, longitudinal information is an essential component of several of the methods, where long stretches of biopolymer are required to obtain sufficient information for species identification, genome scaffolding or analysis of structural variants.

Therefore, there is a need for methods with improved resolution that allow for more detailed and accurate analysis of observed spatial arrangements and patterns.

### SUMMARY OF THE INVENTION

The present invention relates to and includes methods and compositions for biopolymer analysis

One aspect of the present disclosure relates to grafting a linearized biopolymer, either as such or with a specific marker bound to the linearized biopolymer, onto a polymeric matrix. This polymeric matrix is then increased in size. This increase in size occurs in the dimension of the linear biopolymer, but may extend over several dimensions. As size increases according to certain models, site-specific markers on the grafted biopolymer or grafted associated markers retain their relative order, and/or distance patterns can be reconstructed. Measuring the order and/or distance on the polymeric matrix in its increased size yields improved order and/or distance resolving power, allowing the underlying biopolymer structure and identity to be analyzed with increased resolution and accuracy.

In one aspect, the invention also relates to the grafting of the biopolymer onto a hydrogel, wherein size increase is obtained by swelling of the hydrogel by addition of water.

The present invention is well suited for the analysis of polynucleotides, such as DNA and RNA and polypeptides.

Other aspects of the invention will be apparent from the description and examples below, and may be summarized by the following numbered embodiments.

1) A biopolymer analytical method comprising;
1. Grafting the combination of a linearized biopolymer and biopolymer site-specific markers onto a polymer
2. Magnification of the spatial dimensions of the cross-linked polymer along the linearization axis
3. Obtaining position information on site-specific labels

### BRIEF DESCRIPTION OF THE DRAWINGS

Drawing 1 is a schematic representation of the invention

Drawing 2 is a schematic representation of an embodiment of the invention

Drawing 3 schematic representation of one embodiment of the invention

Drawing 4 are some of the chemical compounds as used in the invention

### DEFINITIONS

The following terms and associated definitions are used in this text.

“Stochastic” a random probability distribution or pattern that can be statistically analyzed but cannot be precisely predicted

“Optical reading” here means a method that uses light signals to read specific information about the sample.

“Biorthogonal” here means chemical reactions that can be used in biological systems, in which one reactive group is specifically linked to another reactive group: without side reactions; in neutral, aqueous solution; and under additional conditions compatible with the biological system. Selective reaction between bioorthogonal binding partners can minimize side reactions with other binders, biological compounds, or other non-complementary bioorthogonal binders or non-complementary bioorthogonal functional groups. Bioorthogonal functional groups of bioorthogonal binders include, but are not limited to, an azide and alkyne for the formation of a triazole via Click-chemistry reactions, trans-cyclooctene (TCO) and tetrazine (Tz) (e.g., 1,2,4,5-tetrazine), and others. The binders useful in the present disclosure may have high reactivity with the corresponding binder, so that the reaction proceeds rapidly.

The term “complementary” used here refers to the hybridization or base pairing between nucleotides or nucleic acids, as, for example, between the two strands of a double-stranded DNA molecule or between an oligonucleotide primer and a primer binding site on a single-stranded nucleic acid to be sequenced or amplified. Complementary nucleotides are generally A and T (or A and U), or C and G. Two single-stranded RNA or DNA molecules are said to be complementary when the nucleotides of one strand, optimally aligned and compared and with appropriate nucleotide insertions or deletions, form pairs with at least about 80% of the nucleotides of the other strand, usually at least about 90% to 95%, and preferably from about 98 to 100%. Complementarity also occurs when an RNA or DNA strand hybridizes to its complement under selective hybridization conditions. Characteristically, selective hybridization occurs when there is at least 65% complementarity over a stretch of at least 14 to 25 nucleotides, preferably at least 75%, and preferably at least 90% complementarity.

The term “affinity ligand” refers to a molecule capable of binding to a specific molecule with a specific affinity, and in the present invention the term refers to molecules capable of selectively binding to a protein or aptamer. Note that the affinity ligand may be referred to simply as “ligand.”

The terms “binding” and “binder” refer to a chemical or biological agent that specifically binds to a target (e.g., a targeted biomolecule), forming a stable association between the binder and the specific target. “Stably associated” or “stable association” refers to a molecule being bound to or otherwise associated with another molecule or structure under standard physiological conditions. Bonds may include covalent bonds and noncovalent interactions, such as, but not limited to, ionic bonds, hydrophobic interactions, hydrogen bonds, van der Waals forces (e.g., London dispersion forces), dipole-dipole interactions, and the like. A targeting agent may be a member of a specific binding pair, such as, but not limited to: member of a receptor/ligand pair; a ligand-binding portion of a receptor; member of an antibody/antigen pair; an antigen-binding fragment of an antibody; a hapten; member of a lectin/carbohydrate pair; member of an enzyme/substrate pair; biotin/avidin

The term “contact” or “contact” refers to the process of bringing at least two different species into vicinity so that they can interact with each other, as in a non-covalent or covalent binding interaction or binding reaction. However, the resulting complex or reaction product may be produced directly from an interaction or reaction between the added reagents or from an intermediate of one or more of the added reagents or compositions, which may be produced in the contact mixture.

The term “positional information” refers to position values in a coordinate system. The presence and combination of such values create spatial patterns, which can be interpreted to obtain information about the system.

The term “site-specific” refers to processes, binding events, reactions, “docking” events and the like that occur as a function of the presence or absence of specific combinations of biomonomers. Examples include reactions to specific nucleobases, amino acids and combinations thereof. Sites may consist of specific combinations of nucleobases, amino acids and combinations thereof. Such combinations may comprise any number of monomers, but preferably between 2 and 20.

The term “linker,” “linked,” or “coupling” refers to a chemical part that binds two parts together, such as a connection of the present disclosure to a biological material that targets a specific cell type, such as a cancer cell, another type of diseased cell, or a normal cell type. The coupling may be via covalent bonds, ionic bonds, hydrophobic interactions, hydrogen bonds, van der Waals forces (e.g., London dispersion forces), dipole-dipole interactions, and the like. The coupling may be a direct coupling between the two constituent parts being coupled, or an indirect one, such as via a linker. Linkers useful in embodiments of the present disclosure include linkers having 30 carbon atoms or less in length. In some embodiments, linkers are 1-15 carbon atoms in length, such as 1-12 carbon atoms, or 1-10 carbon atoms, or 5-10 carbon atoms in length. Representative linkers may have 1 to 100 connecting atoms, and may include, but are not limited to, ethylene oxide groups, amines, esters, amides, carbamates, carbonates, and ketone functional groups. For example, linkers may have 1 to 50 bond atoms, or 1 to 30 bond atoms. Other types of linkers may also be used in embodiments of the present disclosure.

The terms “binder” or “binder” refer to a process or agent with a functional group capable of forming a covalent bond with a complementary functional group of another binder in a biological environment. Binding between binders in a biological environment may also be referred to as bioconjugation. Representative binders include, but are not limited to, an amine and an activated ester, an amine and an isocyanate, an amine and an isothiocyanate, thiols for the formation of disulfides, an aldehyde and an amine for enamine formation, an azide for the formation of an amide via a Staudinger ligation. Binders also include bioorthogonal binders, that is, binders with bioorthogonal functional groups.

The “nucleic acids” or “polynucleotides” of the invention include, but are not limited to, ribonucleic acids (RNAs), deoxyribonucleic acids (DNAs), threosenucleic acids (TNAs), glycolnucleic acids (GNAs), peptide nucleic acids (PNAs), closed nucleic acids (LNAs, including LNA with a β-D-ribo configuration, α-LNA with an α-L-ribo configuration (a diastereomer of LNA), 2′-amino-LNA with a 2′-aminofunctionalization, and 2′-amino-α-LNA with a 2′-aminofunctionalization), ethylene nucleic acids (ENA), cyclohexenyl nucleic acids (CeNA) or hybrids or combinations thereof.

The term “oligonucleotide,” as used in the invention, refers to a short nucleic acid molecule from about 8 to about 50, possibly up to about 90 nucleotides in length, natural or synthetic, that can act as a starting point of complementary nucleic acid hybridization. If desired, the oligonucleotide itself can also be labeled by including a compound that can be detected by spectroscopic, photochemical, biochemical, immunochemical or chemical means. Useful labels include fluorescent dyes, electron-dense reagents, biotin, or small peptides for which antisera or monoclonal antibodies are available.

A “reactive group” is a chemical moiety that can react with a chemical partner moiety to form a covalent or non-covalent bond. A group can be considered a reactive group by virtue of its high reactivity with a single partner molecule, a pair of partner molecules, or by virtue of its reactivity with many partners.

“Nucleic acid mapping” refers to a process in which sequence-specific markers are inserted into a polynucleotide, and the spacing information between these markers provides information about the genetic makeup of the polynucleotide. DNA mapping can involve all polynucleotides in a sample, including but not limited to genomic DNA, plasmid DNA, mRNA, tRNA and genomic RNA. In the case that the polynucleotide is DNA, nucleic acid mapping is referred to as DNA mapping.

### DETAILED DESCRIPTION OF THE INVENTION

The present invention will be described by particular embodiments and with reference to particular drawings, but the invention will not be limited in this respect, but only by the claims. Further, the terms first, second and the like will be used in the description and in the claims to distinguish between similar elements and not necessarily to describe an order, whether temporal, spatial, rank or in any other manner. It is to be understood that the terms so used are interchangeable under appropriate circumstances and that embodiments of the invention described herein may also operate in different sequences than those described or illustrated herein.

It should be noted that the term “consisting of” used in the claims should not be interpreted as being limited to the means listed below; it does not exclude other elements or steps. Thus, the term is to be interpreted as specifying the presence of the said features, integers, steps or components, but does not exclude the presence or addition of one or more other features, integers, steps or components, or groups thereof. Thus, the scope of the expression “a device comprising parts A and B” should not be limited to devices comprising only parts A and B. It means that, as far as the present invention is concerned, the only relevant parts of the device are A and B.

Reference in this specification to “an embodiment” or “an embodiment” means that a particular feature, structure or characteristic described in connection with the embodiment is included in at least one embodiment of the present invention. Thus, the expressions “in an embodiment” or “in an embodiment” appearing in various places in this specification do not necessarily all refer to the same embodiment, but may all refer to the same embodiment. Further, the particular features, structures or characteristics may be combined in any suitable manner, as would be obvious to one of ordinary skill in the art from this disclosure, in one or more embodiments.

Similarly, it should be understood that in describing exemplary embodiments of the invention, various features of the invention are sometimes grouped together in a single embodiment, figure or description thereof, for the purpose of streamlining the disclosure and assisting in understanding one or more of the various inventive aspects. However, this method of disclosure should not be interpreted to mean that the claimed invention requires more features than those expressly disclosed in each claim. On the contrary, as can be seen from the following claims, the inventive aspects lie in less than all the features of a single aforementioned disclosed embodiment. Thus, the claims subsequent to the detailed description are hereby expressly incorporated into this detailed description, each claim standing alone as a separate embodiment of this invention.

The description provided herein sets forth numerous specific details. However, it is understood that embodiments of the invention can be implemented even without these specific details. In other instances, known methods, structures and techniques are not shown in detail so as not to obscure the understanding of this description.

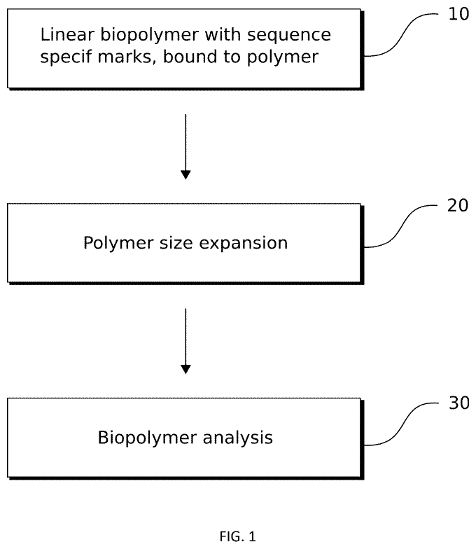

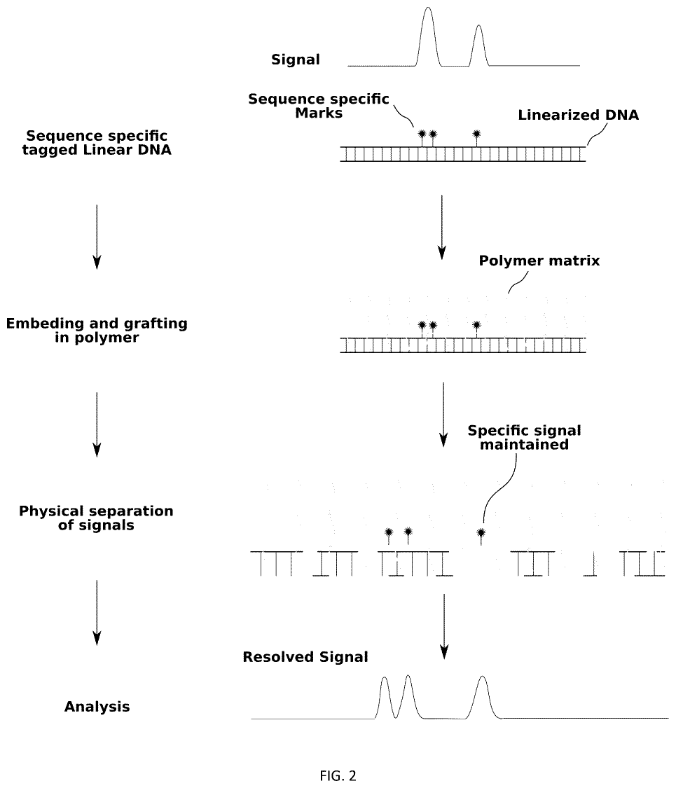

The method of the invention includes obtaining a linearized biopolymer in or on a polymeric matrix, where the polymeric matrix contributes to analysis of the linearized biopolymer An embodiment of the described method 100 is shown in FIG. 1 and includes 3 different steps, [10, 20, 30], which can be divided as follows
A. Obtaining a linearized biopolymer marked at specific sites and bound to a polymeric matrix
B. Increasing the dimensions of the polymer, and this at least in line with the linearized biopolymer
C. Analysis of the signal

For the methods of the invention, the first step itself may comprise several sub-steps. For example, a biomolecule may react in a site-specific manner to place labels at known sites within the polymer, followed by linearization of the biomolecule together with the bound labels. Alternatively, the biomolecule may be linearized first, followed by specific binding at selected sites within the biomolecule.

Linearization of the biomolecule can be performed by linearization on a surface or in a flow system. Examples of linearization include linearization in on surfaces (Kaykov et al. Sci Rep 6, 19636 (2016) or in flow systems (Kim et al, Lab Chip, 2011,11, 1721-1729).

Increasing the size of the polymer can be achieved by merely physically stretching the polymer, by applying a force. Since the direction of the linearized biopolymers is known, the direction of the force can be consistent with the direction of the biopolymer.

In a specific embodiment, the polymer matrix forms an swellable matrix. A well-known example of such an swellable matrix is hydrogels, which consist of a polymer mesh and an aqueous solution. With further stimulation, the physical dimensions of the hydrogel increase. Traditional stimuli that trigger a hydrogel reaction are pH, temperature, solvents and ionic strength. Dimensions 4-20 times larger can easily be achieved with different hydrogel recipes.

In yet another embodiment, the swelling is limited in some of its dimensions by a holder. This may force the gel to limit swelling in a preferred direction, enhance swelling in a preferred direction, or ensure proper handling of the gel.

Preferably, the size expansion of the polymer follows predetermined patterns so that the original biological information can be traced with high resolution. This pattern may be a linear size expansion. For applications where only the presence and sequence of specific sites is important, this preknown expansion pattern is of less importance. In a specific embodiment, a measurement is included in the workflow to analyze the size expansion profile.

Groups capable of effecting such grafting onto the polymer matrix include, but are not limited to, vinyl or vinyl monomers such as styrene and its derivatives (e.g., divinylbenzene), acrylamide and its derivatives, butadiene, acrylonitrile, vinyl acetate, maleimides, alkynes, epoxides, aldehydes or acrylates and acrylic acid derivatives.

In a further embodiment, the methods of the inventions may include a step that breaks the biopolymer after grafting the specific signals onto the polymeric matrix. This may be beneficial for the uniformity of size gain, since the covalent bonds in the biopolymer may hinder local size gain if not broken. Examples of such steps include a UV treatment to disrupt the polynucleotide chains or an enzymatic step, with a nuclease or proteinase enzyme.

In embodiments of the invention, site-specific labeling of DNA is a key element of the methods. Such sequence-specific labeling of DNA is a rapidly developing field discussed by Gottfried A. et al. “Sequence-specific covalent labeling of DNA”, Biochemical Society Transactions, 39(2), 623-628” and the sequence-specific methods described herein are incorporated by reference. Enzyme-specific methods include labeling with methyltransferase enzymes, nicking enzymes, polymerases and CRISPR-CAS methods. Chemical methods include the use of sequence-specific ligands, which can be small molecules or oligomers.

In general, DNA nucleobases are buried in the DNA helix, with only limited availability for chemical modification on the nucleobase. However, in a specific embodiment of the invention, DNA is linearized on a surface using molecular combing. This molecular combing causes partial melting of the DNA, making nucleobases more accessible for chemical reactions. For example, bisulfite-mediated transamination of cytosines is able to generate a DNA signature within a polymeric matrix. In such an embodiment, it may be advantageous to graft the DNA onto the polymeric matrix in a non-specific manner after linearization. The polymeric matrix can then be subjected to the required chemicals and solutions without the risk of the DNA becoming detached from its position on the surface.

The methods of the invention are readily translatable to some existing DNA mapping approaches, where sequence-specific markers are applied to DNA prior to analysis. By further modifying this DNA with a polymerizable moiety, the DNA can be subjected to grafting with a polymer matrix, the polymer can be expanded, and DNA mapping can occur with improved resolution.

A particular property of DNA can be used for efficient signal generation or incorporation of monomeric molecules. Agents that bind to the major or minor groove of DNA can generate a sequence-specific signal (Dervan et al., Proc Natl Acad Sci U S A. 2006; 103(4):867.), serve as a way to insert monomeric units along the DNA backbone or a combination of both.

In another embodiment, DNA is denatured to obtain single-stranded DNA, followed by hybridization with random oligomers. The oligomers may then be modified oligomers containing polymerizable groups or tags, or a combination thereof.

In some embodiments, the method further comprises detecting a relative distance between labels on the linearized polynucleotide, thereby obtaining a barcode of a portion of the genomic information. This relative distance can be obtained by fluorescence microscopy.

Clearly, the linearized biomolecules do not have to be located in the polymeric matrix, but the sequence-specific markers only have to be bound or transferred to the polymeric matrix. Thus, the biopolymers may be located on or against the polymeric matrix, and the sequence-specific marker is bound to or in the polymeric matrix.

Thus, in such embodiments of the methods of the invention, the signal for analysis no longer becomes a direct signal of the biomolecule, but of its identity. Thus, the biomolecule no longer needs to maintain its integrity after binding of the specific markers to or in the polymeric matrix.

Sequence-specific marking of RNA can be achieved by sequence-specific interactions or direct reactions with specific nucleobases.

Sequence-specific interactions, or interactions that depend on the sequence of more than one nucleobase, that can be used for the methods of the invention are, for example, complementary hybridization probes or sequence-specific enzymes.

For these embodiments, the hybridization probes can preferably be selected for a high melting temperature so that the secondary and tertiary structures of the RNA are disrupted. Examples of such hybridization probes with favorable binding include peptide nucleic acids (PNA), closed nucleic acids (LNA) and other non-natural polynucleotides.

The length of the hybridization probes is chosen depending on the application, but is preferably between 4 and 400 nucleotides.

Importantly, in the methods described in the invention, the sequence-specific signature is transposed onto a polymeric matrix. The integrity of the biomolecule need not be maintained to analyze the sequence-specific signature.

To facilitate linearization, agents that disrupt secondary and tertiary RNA structure that would otherwise interfere with the linearization process can be added to the linearization mixture. Such agents can be random oligonucleotides (e.g., random hexamers or nonamers), agents that break hydrogen bonds (e.g., formamide, urea) or charge neutralizing salts.

Functionalization of RNA species according to the methods of the invention can also be achieved by targeting specific nucleobase interactions to RNA. For example, bisulfite-mediated cytosine transamination rapidly and completely converts all cytosines to an amine-exchanged variant. In this way, all cytosines in an RNA can be functionalized with both signaling molecules and monomeric functionalities. Therefore, the sequence-specific signal generated after binding to the polymer matrix and expansion is a linear image of the cytosine content of the RNA. Guanine labeling can be accomplished by the action of reagents such as the electrophilic nitrogen mustards, platinum reagents, which preferentially react with the nitrogen of guanine nucleobases.

In a specific embodiment, nucleobase-specific labeling is combined with hybridization probes. For example, bisulfite-mediated transamination is hindered when portions of the RNA are hybridized. As such, RNA signals can be prepared that combine both a signal at non-hybridized sites, reflecting the presence of cytosine, and a “dark signal” at the position of specific hybridization probes. Alternatively, an initial cytosine transamination is performed in the presence of hybridization probes, and signal is generated in one channel (e.g., one color), with hybridization probes in a second channel (e.g., a different color).

A unique advantage of the methods of the invention is that the combination of sequence-specific marking of RNA with an extended polymer matrix brings RNA within the reach of high-resolution genomic mapping and Fiber FISH, methods previously blocked by the limited size of most RNA. No other methods exist that allow direct imaging and identification of mRNA and rRNA.

In a specific embodiment of the invention, there are hybridization or affinity probes in the polymerization mixture, polymerization center or linearization center that contribute to the linearization of the biopolymer. These probes can also carry a signal function. A possible example is a hybridization probe for RNA, such as a poly-T tail for hybridization of poly-A containing RNA.

In particular, direct imaging of rRNA and its genetic signature will have extensive applications in species identification, lineage and phylogenetics, and analysis of complex mixtures, or microbiomes. Currently, these signals are measured indirectly through analysis of the DNA encoding the rRNA, by targeted amplification of the genes followed by sequencing, microarray analysis, qPCR and other methods.

In specific embodiments, the method is extended to mapping Proteins. By marking specific amino acid side chains with signals, linearizing the polypeptide and binding it to the polymer matrices, a linear signal can be obtained. This signal corresponds to a rough representation of the overall protein sequence. However, this rough representation of the sequence of amino acids contains sufficient information to establish the identity of the protein.

It is obvious to one trained in the art that diffraction-limited microscopy methods can further increase the resolution of the genetic information obtained.

The methods of the invention have broad application potential in the fields of biotechnology, genomics, medicine and beyond.

In particular, the methods enable unprecedented resolution in approaches to genomic mapping, where sequence-specific signatures of linearized DNA are used to establish DNA identity and origin. Such genomic mapping is used in species identification, genome mapping, genomic and genetic analysis and analysis of structural variants. Here, the sequence-specific signature is directly related to length, as the DNA piece becomes more unique as a function of length. In current genomic mapping approaches, sequence-specific elements are targeted at 4 to 8 base pairs, and DNA pieces are longer than 10 kbp to MBp. The extension of the genetic signature leads to greater resolving power of the genetic signature, and therefore to more specific attribution.

So far, no genetic mapping methods have been extended to RNA species. This is largely attributed to the length requirements mentioned above. With mRNA several hundred to several thousand bases long, the signature obtained in genomic mapping approaches would not contain sufficient information for further use. However, building on the methods of the invention, high-density marking of RNA can be physically separated by polymer extension to yield a useful trace. As such, genomic mapping of mRNA from tissues and cells becomes feasible. rRNA from various organisms and mixtures thereof can be directly visualized, studied and assigned.

These methods can also be applied to other enzymes and proteins that bind to specific sites on a polynucleotide or to specific states of a polynucleotide. Examples of such enzymes and proteins that bind to polynucleotides in a sequence-specific manner beyond methyltransferases are transcription factors or proteins that contain DNA-binding domains such as Helix-turn-helix, Zinc finger, Leucine zipper, Winged helix, Winged helix-turn-helix, Helix-loop-helix, HMG-box, Wor3 domain, OB-fold domain, Immunoglobulin fold, B3 domain or TAL effector. A specific case of DNA-binding proteins are proteins guided by RNA. For example, Cas9 can be used as an adaptable RNA-guided DNA-binding platform, and when combined with ligands as described in this invention, be capable of targeted DNA modification.

Another example are enzymes that bind to accessible chromatin, transfer functionality to said chromatin and allow specific analysis of chromatin accessibility and its use in genetic analysis.

A specific embodiment is the use of the methods of the inventions for the analysis of epigenetic and post-translation markers on polynucleotides and polypeptides. Examples include site-specific labeling such as DNA hydroxymethylation, DNA carbonylation, specific RNA bases such as pseudouridine or protein modifications such as prenylation or phosphorylation. Specific chemical, enzymatic or chemoenzymatic approaches exist for transferring functionality to these specific sites, and when combined with linearization and grafting onto a polymeric matrix, the extension of the signal can enhance the analysis of these marks.

Further embodiments of the inventions may replace the enzyme with synthetic macromolecules capable of recognizing specific docking positions on the biomolecule. An example of such macromolecules may be an aptamer. Another example of such macromolecules may be an antibody. Specific antibodies and aptamers have been described in the literature for genetic features such as DNA methylation and specific sequence combinations, and can therefore be readily incorporated into the methods of the invention.

In each method described above, (a) the biopolymer labeling reactions can be carried out in the same reaction vessel, or (b) biopolymer from the sample can be divided into aliquots and added to different reaction vessels, carrying out biopolymer labeling reactions in each reaction vessel. The different reactions can then be recombined or analyzed independently.

In the kits of the invention, at least one marker entity may be a fluorophore or a molecule to which a fluorophore may be attached.

Each of the kits of the invention may further include at least one affinity chromatography column.

Each of the kits of the invention may further include at least one buffer.

Each of the kits of the invention may further include one or more other enzymes for performing reactions required in methods of the invention. The enzymes may include DNA methyltransferases, DNA nickase or a DNA polymerase.

Particularly useful are labels that are optically detectable. Examples include chromophores, luminophores and fluorophores. Fluorophores are particularly useful and include, for example, fluorescent nanocrystals, quantum dots, fluorescein, rhodamine, tetramethylrhodamine, eosin, erythrosine, coumarin, methyl-coumarins, pyrene, malachite green, Cy3, Cy5, stilbene, Lucifer yellow, Cascade blue, Texas red, Alexa dyes, SETA dyes, Atto dyes, phycoerythine, bodipy and analogues thereof. Useful optical probes are described in Haugland, Molecular Probes Handbook, (Eugene, Oreg.) 6th Edition; the Synthegen catalog (Houston, Tex.), Lakowicz, Principles of Fluorescence Spectroscopy, 2nd Ed., Plenum Press New York (1999), or WO 98/59066; WO 91/06678 or US Pat. Appl. Publ. No. 2010/0092957 A1, all of which are incorporated herein by reference. Optical labels offer the advantage of rapid, relatively non-invasive detection.

Other labels, some of which are non-optical labels, can be used in various applications of the methods and compositions described here. Examples include, without limitation, an isotopic label such as a naturally nonredundant radioactive or heavy isotope; magnetic substance; electron-rich material such as a metal; electrochemiluminescent label such as Ru(bpy); or a part that can be detected based on a nuclear magnetic, paramagnetic, electrical, charge-mass, or thermal characteristic. Labels can also include magnetic particles or optically encoded nanoparticles. Such labels can be detected using appropriate methods known to those knowledgeable in the field. For example, a charged label can be detected with an electrical detector such as those used in commercially available sequencing systems from Ion Torrent (Guilford, Conn., a subsidiary of Life Technologies) or detection systems described in US Pat. App. Publ. Nos. 2009/0026082 A1; 2009/0127589 A1; 2010/0137143 A1; and 2010/0282617 A1, all of which are incorporated herein by reference. It will be clear that for some applications, a nucleotide analog need not have a label.

In one embodiment, the reporter is an oligonucleotide. By specific hybridization with complementary oligonucleotides, this reporter can serve as a barcode for the biomolecule originally present at the named site. The complementary oligonucleotides may be fluorescently labeled (i.e., they are conjugated to a fluorophore). Fluorophores conjugated to imager strands of different nucleotide sequence may be identical to each other, or they may have an emission profile that overlaps or does not overlap with that of other fluorophores. The fluorescently labeled imager strand may contain at least one fluorophore. Both reporter oligonucleotides and signaling oligonucleotides may possess a hairpin secondary structure.

In one embodiment, the complementary oligonucleotide hybridization is transient, with binding times in a sufficiently long time scale to cause a “blinking” event.

In yet another embodiment, the reporter group comprises multiple discrete signals.

For some embodiments of the invention, some form of signal amplification may be useful. Such signal amplification provides a good readout of low abundance signals or allows the technique to be used in less demanding analytical setups, since the need for expensive detectors and sensitive readout is less. Examples of such amplification methods can be hybridization-based, such as Hybridization-Chain-Reaction (HCR), SABER or Rolling Circle amplification. Depending on the type of amplified signal, this amplification can be performed before or after swelling. Another example of signal amplification is the use of antenna dyes, which amplify the signal generated by one dye by harvesting excitation light.

Another type of label that may be useful is a secondary label that is indirectly detected, such as via interaction with a primary label, binding to a receptor or conversion into a detectable product by an enzyme catalyst or other substance. An example of a secondary label is a ligand such as biotin or analogues thereof, which can be detected via binding to a receptor such as avidin, streptavidin or analogues thereof. Other useful ligands include epitopes that can bind to receptors such as antibodies or active fragments thereof, and carbohydrates that can bind to receptors such as lectins. The receptors can be labeled, for example with an optical label, so that they can be detected. In special applications, the ligand can be attached to a nucleotide analogue in such a way as to reduce or prevent its affinity for a receptor. The release of the ligand can then be detected based on the affinity of the ligand for its respective receptor when it is detached from the nucleotide analogue. The ligand can be further coupled to a blocking group or can itself act as a blocking group, as explained above more generally for labeling groups. Thus, removal of the ligand from a nucleotide analogue can function to unblock the nucleotide analogue and provide a detectable event.

### EXAMPLES

### Example 1

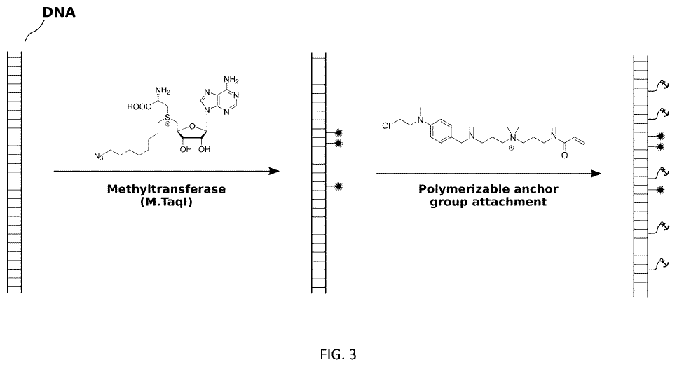

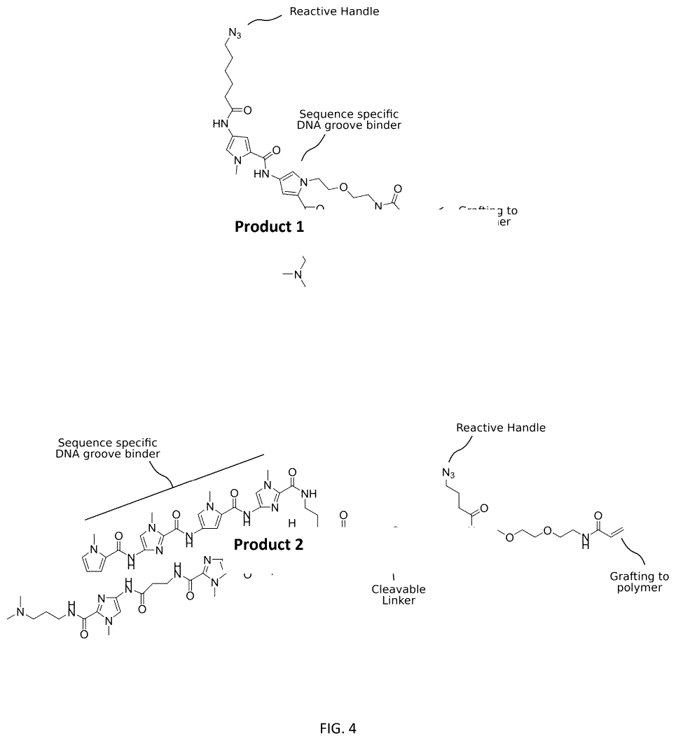

Lambda phage DNA is incubated with in the presence of Product 1 (FIG. 4) at 55° C. for 60 minutes. The DNA is deposited on a polymer surface in accordance with literature examples (Kaykov et al., Sci Rep 6, 19636 (2016)). The surface is covered with a polymerization mixture (aqueous solution of sodium acrylate, bisacrylamide, TEMED and APTS, in line with standard literature recipes), and polymerization is performed for 40 minutes. The polymeric gel is gently detached from the substrate and allowed to incubate for 30 minutes at 37° C. in a solution containing TAMRA-DBCO (Jena Biosciences) in PBS. The gel is washed extensively with PBS followed by swelling in double distilled water overnight. The swollen gel is mounted on a coverslip and imaged. The linear features are analyzed in accordance with the literature (Torche PC, PLoS ONE 12(6):e0179041) to reveal the sequence-specific signal.

1. A method for characterizing biopolymers, the method comprising:
i) grafting the combination of a linearized biopolymer and biopolymer site-specific markers onto a polymer;
ii) magnifying a spatial dimensions of the polymer along a linearization axis; and
iii) obtaining positional information from place-specific labels.

2. The method according to claim 1, wherein increasing the spatial dimensions of the polymer is obtained by a swelling of a hydrogel.

3. The method according to claim 1, wherein increasing the spatial dimensions of the polymer is obtained by applying a force to the polymer.

4. The method according to claim 1, wherein the biopolymer is a polynucleotide.

5. The method according to claim 2, wherein the biopolymer is DNA.

6. The method according to claim 2, wherein the biopolymer is RNA.

7. The method according to claim 2, wherein the positional information is obtained by fluorescence microscopy.

8. The method according to claim 7, wherein it is applied to identify an origin of the biopolymer.

9. The method according to claim 7, wherein applied in cellular analysis.

10. The method according to claim 7, wherein applied in an identification or a treatment of a disease.
